(19) United States
(12) Patent Application Publication
(10) Pub. No.: US 2008/0161570 A1
(43) Pub. Date: Jul. 3, 2008

(54) Processes for Synthesizing Quaternary 4,5-Epoxy-Morphinan Analogs and Isolating their N-Stereoisomers

(75) Inventors: Julio Perez, Tarrytown, NY (US); Amy Qi Han, Hockessin, DE (US); Alfred A. Avey, Eugene, OR (US)

Correspondence Address: KELLEY DRYE & WARREN LLP, 400 ALTLANTIC STREET , 13TH FLOOR, STAMFORD, CT, 06901, US

(73) Assignee: Progenerics Pharmaceuticals, Inc., Tarrytown, NY (US)

(21) Appl. No.: 11/942,292

(22) Filed: Nov. 19, 2007

Related U.S. Application Data
(60) Provisional application No. 60/867,103, filed on Nov. 22, 2006.

(51) Int. Cl. C07D 489/08

(52) U.S. Cl. 546/45; 546/46

(57) ABSTRACT

Methods for the synthesis of 4,5-epoxy-morphinaniums using dimethyl formamide and resolution of the diastereomeric products by means of HPLC.

This application claims priority to U.S. Provisional Patent Application 60/867,103, which is incorporated herein in its entirety.

### BACKGROUND OF THE INVENTION

1. Field of the Invention

The present invention generally relates to processes for forming quaternary 4,5-epoxy-morphinan analogs, synthetic methods for their preparation, pharmaceutical preparations comprising the same, and methods for their use. It also generally relates to methods for isolating the N-stereoisomers of the synthesized quaternary 4,5-epoxy-morphinan analogs.

2. Description of the Related Art

A number of side-effects produced by opioid agonists are believed to be of central origin. In order to avoid such side effects, peripheral opioid agonists and antagonists that do no cross the blood-brain barrier into the central nervous system have been proposed and developed.

WO 2004/029059 discloses N-quaternary hydromorphone agonists wherein the nitrogen carries a methyl substituent and a C₁-C₆ substituent. Such compounds are asserted to provide potent mu-agonist activity, but to not cross the blood-brain barrier, thereby reducing opioid agonist CNS side effects. Similarly, WO 2004/043964 discloses N-methyl quaternary derivatives of antagonistic morphinan alkaloids, naltrexone and naloxone, as potent antagonists of the mu receptor, which because of their ionic charge do not traverse the blood brain barrier into the central nervous system. It is suggested that such quaternary derivatives do not block the pain relieving activity of agonistic opioids (or the endogenous opioid compounds produced naturally) when the two are concomitantly administered exogenously.

Synthesis of a number of morphinanium compounds pose special problems particularly when taking into account the reactivity of certain substituents, and rearrangements, on the compounds. For example, quaternization of oxymorphone structures while seemingly trivial has been found to be difficult.

Goldberg et al., U.S. Pat. No. 4,176,386, teaches the use of methyl halide and dimethylsulfate alkylating agents to convert tertiary N-substituted noroxymorphone compounds and O-substituted tertiary noroxymorphone to quaternary compounds.

Cantrell and Halvachs, WO 2004/043964, disclose processes for the preparation of quaternary n-alkyl morphinan alkaloid salts from tertiary N-substituted morphinan alkaloids using alkyl halides in an anhydrous solvent system. The anhydrous solvent system comprises an aprotic dipolar solvent with the aprotic dipolar solvent constituting at least 25 wt % of the solvent system. They further disclose a process for separating a liquid mixture containing a 3-alkoxymorphinan alkaloid and a 3-hydroxymorphinan alkaloid comprising contacting the mixture with a strong base converting the 3-hydroxy morphinan to a salt, and then precipitating the salt but not the 3-alkoxymorphinan alkaloid from the liquid. The salt precipitate is then separated from the 3-alkoxymorphinan alkaloid.

Schmidhammer et al., U.S. Appl. Pub. No. 2005/0182258, discloses a number of processes for forming quaternary ammonium salts of morphinan compounds which may have substituents at the C-3 and C-14 positions of the backbone.

In one process of the Schmidhammer reference, the production of quaternary morphinan derivatives starts from thebaine. Thebaine is converted to a 14-hydroxycodeinone by reacting the thebaine with a reactant to in the presence of a strong base which is chosen to react at the R-3 position of the backbone. Reactant compounds cited include dialkylsulphates, fluorosulphonic acid alkylesters, alkylsulphonic acid alkylesters, arylsulphonic acid alkylesters, alkylhalogenides, aralkylhalogenides, alkylsulphonic acid aralkylesters, arylsulphonic acid aralkylesters, arylalkenylhalogenides, chloroformic acid esters and similar compounds in solvents such as tetrahydrofuran, 1,2-dimethoxyethane, diethylether or similar compounds. Strong bases cited include n-butyllithium, lithium diethylamide, lithium di-isopropylamide or similar compounds. Such reaction is said to be carried out at low temperatures (−20° C. to −80° C.). Resulting compounds may be converted into the corresponding 14-hydroxy by carrying out an addition reaction with performic acid, m-chloroperbenzoic acid at temperatures between 0° and 60° C. The 14-hydroxy is said to be able to be modified by reaction in sequence with dialkylsulphates, alkylhalogenides, alkenylhalogenides, alkinylhalogenides, arylalkylhalogenides, arylalkenylhalogenides, arylalkinylhalogenides or chloroformates in solvents such as N,N-dimethylformamide (DMF) or tetrahydrofuran (THF) in the presence of a strong base such as sodium hydride, potassium hydride or sodium amide. These compounds then may be reduced by using catalytic hydrogenation via a catalyst such as Pd/C, PdO, Pd/Al₂O₃, Pt/C, PtO₂, Pt/Al₂O₃ in solvents comprising alcohol, alcohol/water, or glacial acetic acid. The N-methyl is indicated to be replaceable by means of chloroformates or bromocyanogens in solvents such as 1-2-dichloromethane or chloroform and reaction with the appropriate leaving group followed by splitting by reflux heating in alcohols or by the addition of hydrogen halogenides or halogens followed by reflux x heating in alcohol. The N-alkylation of the compounds are indicated to be effectuated by reacting the desired side group in a solvent such as dichloromethane, chloroform or N,N-dimethylformamide in the presence of a base such as sodium bicarbonate, potassium carbonate, or triethylamine. Ether splitting with boron tribromide at 0° C., 48% hydrobromic acid (reflux heating), with sodium alkanthiolates (in a solvent such as N,N-dimethylformamide) can be used to form a phenolic ring. 3-O alkylation is said to be achievable by alkylhalogenides, dialkylsulphates, alkenylhalogenides, alkinylhalogenides, cycloalkylalkylhalogenides, cycloalkylalkenylhalogenides, arylalkylhalogenides, arylalkenylhalogenides, arylalkinylhalogenides or similar in solvents such as dichloromethane, chloroform, acetone or N,N-dimethylformamide in the presence of a base such as sodium bicarbonate, potassium carbonate, or triethylamine. 3-O acylation is said to be achievable with carboxylic acid halogenides, carboxylic acid anhydrides or similar in solvents such as dichloromethane, chloroform, acetone, N,N-dimethylformamide, or pyridine.

An alternative process set forth in the Schmidhammer reference starts with substituted 14-hydroxy substituted N-tertiary hydroxymorphonone. Such compound is reacted in the presence of methane sulphonic acid with ethylene glycol (as reagent and solvent) to form a dioxopentyl ring. A protective group is introduced to protect the 3-hydroxy group, such as for example benzyl, trityl or silyl by means of 3-O-benzylation, 3-O-tritylation or 3-O-silylation of the compounds of the formula (XIII) with benzyl halogenides, trityl halogenides, trialkyl halogen silanes in solvents such as dichloromethane, chloroform, acetone or N,N-dimethylformamide in the presence of a base such as sodium bicarbonate, potassium carbonate, or triethylamine. The resulting 14-hydroxy compounds are then reacted with dialkylsulphates, alkylhalogenides, alkenylhalogenides, alkinylhalogenides, arylalkylhalogenides, arylalkenylhalogenides, arylalkinylhalogenides or chloroformates in solvents such as N,N-dimethylformamide (DMF) or tetrahydrofuran (THF) in the presence of a strong base such as sodium hydride, potassium hydride or sodium amide. The acidic splitting of the 3-O protective group and the ketal function of the compounds with the formula (XV) is carried out with an acid such as hydrochloric acid in methanol, tetrafluoroboric acid in dichloromethane or trifluoroacetic acid. Alternatively to this, if R₄ is benzyl, it is indicated that through hydrogenolysis of the 3-O-benzyl binding with hydrogen gas in the presence of a catalyst such as Pd/C, PdO, Pd/Al₂O₃, Pt/C, PtO₂, or Pt/Al₂O₃ in solvents such as alcohols, alcohol/water mixtures, or glacial acetic acid, followed by acid hydrolysis of the ketal function at position 6 of the backbone with, for example, methanol and concentrated hydrochloric acid. The resulting compounds may be reacted according to the first scheme described above to form compounds of interest.

The art suggests that isolated stereoisomers of a compound, whether enantiomers or diasteromers, sometimes may have contrasting physical and functional properties, although it is unpredictable whether this is the case in any particular circumstance. Dextromethorphan is a cough suppressant, whereas its enantiomer, levomethorphan, is a potent narcotic. R,R-methylphenidate is a drug to treat attention deficit hyperactivity disorder (ADHD), whereas its enantiomer, S,S-methylphenidate is an antidepressant. S-fluoxetine is active against migraine, whereas its enantiomer, R-fluoxetine is used to treat depression. The S-enantiomer of citalopram is therapeutically active isomer for treatment of depression. The R-enantiomer is inactive. The S-enantiomer of omeprazole is more potent for the treatment of heartburn than the R enantiomer.

The designations “R” and “S” are commonly used in organic chemistry to denote specific configuration of a chiral center. The designations “R” refers to “right” and refers to that configuration of a chiral center with a clockwise relationship of group priorities (highest to second lowest) when viewed along the bond toward the lowest priority group. The term “S” or “left” refers to that configuration of a chiral center with a counterclockwise relationship of group priorities (highest to second lowest) along the bond toward the lowest priority group. The priority of groups is based upon atomic number (heaviest isotope first). A partial list of priorities and a discussion of stereochemistry is contained in the book: The Vocabulary of Organic Chemistry, Orchin, et al. John Wiley and Sons, Inc., page 126 (1980), which is incorporated herein by reference in its entirety. When quaternary nitrogen morphinan structures are produced, such structures may be characterized as R or S.

Synthesis and isolation of select N-stereoisomers may pose harrowing problems. Selective synthesis of one stereoisomer versus another may be desired in order to reduce cost in the production of the desired stereoisomer, and may be necessary when isolation from the other N-stereoisomer is difficult.

Streicher and Wunsch, Synthesis of Enantiomerically Pure Morphan Analogues from α-D-Glucose, 2001 Eur. J. Org. Chem. 115-120 disclose the synthesis of an enantiomerically pure bicyclic morphan analog. The epoxyazocane compound was produced via an intramolecular N/O-acetal formation of amino or amido acetals from methyl glucopyranoside.

Koczka and Bernath, Selective Quaternization of Compounds with Morphine Skeleton, 1967, Acta Chimica Academiae Scientiarum Hungaricae. Tomus, 51: 393-402 suggest that in respect of certain morphine analogs (having an unsaturated cyclohexanone ring) there can be selective synthesis of R and S isomers using methyl iodide or allyl iodide in chloroform at about +4° C. They report with respect to their investigated compound, N-allyl-N-methyl-normorphine, that the substituent coupled to the nitrogen atom in the second instance occupied the axial steric position in the quaternary salt form as the main product.

Iorio and Frigeni, Narcotic agonist/antagonist properties of quaternary diastereoisomers derived from oxymorphone and naloxone, 1984, Eur. J. Med. Chem. 4: 301-303, report that oxymorphone and naloxone, morphinan analogs having a completely saturated cyclohexane ring, when reacted with allyl iodide and methyl iodide, respectively, show a strong degree of axial selectivity. The group reported that when analyzed by ¹H NMR spectroscopy, the presence of the corresponding diastereoisomer was not detected. The authors expressed their belief that such behavior was unexpected in light of other compounds wherein the presence of an axial substituent β to nitrogen is generally associated with decreased preference for an axial approach (they note this is especially true with respect to larger incoming groups). Funke and deGraaf, A ¹H and ¹³C nuclear magnetic resonance study of three quaternary salts of naloxone and oxymorphone, 1986, J. Chem. Soc. Perkin Trans. II 735-738, referencing Ioria et al., report the ¹H and ¹³C NMR data with respect to three N,N-dialkyl-morphinanium chloride derivates (one N,N-diallyl and two N-allyl-N-methyl diastereoisomers).

The research of Koczka and Bernath, Iorio and Frigeni, and Funke and deGraaf, discussed above, relates to stereoselective processes with respect to a small number of morphinans and a small number of reactants. Such studies do not support the hypothesis that stereoselectivity will be seen with respect to the same reactants when reacted with other morphinan structures, nor the same morphinan structures with other reactants. In fact, the present inventors have found in general little stereoisomeric selectivity in processes employed in the past to produce morphinanium analogs.

In addition to the isolation and characterization of each stereoisomer of quaternary narcotic antagonists, it may be of high importance to isolate the particular stereoisomer from impurities in their manufacture. Certain impurities may be formed that may hinder the therapeutic effect of quaternary morphinans and/or may be toxic if present in high enough quantity. Further, regulatory standards may require a high level of purity. It is desirable, therefore, that one have the ability to determine both the stereochemical configuration and purity of the quaternary morphinan. To do this, it may be necessary to identify, isolate and chemically characterize impurities, which then can be used in chromatographic procedures as standards to confirm the purity of the isolated stereoisomer.

There is a need for alternative methods for producing morphinanium analogs. In particular, there is a need for new methods to selectively produce morphinanium analogs in a stereoisomeric form which is associated with a particular desired pharmacological effect.

### SUMMARY OF THE INVENTION

There is provided in embodiments herein processes for forming quaternary 4,5-epoxy-morphinan analogs, synthetic methods for their preparation, pharmaceutical preparations comprising the same, and methods for their use. There is also provided herein methods for isolating the N-stereoisomers of the produced quaternary 4,5-epoxy-morphinan analogs.

Alkyl halides are often used to quaternize the nitrogen of the morphinan ring structure. For example, Cantrell et al., U.S. Patent Public. No. 2006/0014771 discloses the preparation of N-alkyl quaternary derivatives from a ternary alkaloid by contacting the alkaloid with an alkyl halide, comprising about 1 to 8 carbons, in an anhydrous solvent system. The solvent system for N-alkylation is disclosed as an aprotic, dipolar solvent which is anhydrous. The reference lists a number of exemplary aprotic dipolar solvents including dimethyl acetamide, dimethyl formamide, N-methylpyrrolidinone, acetonitrile, hexamethylphosphor-amide (“HMPA”), and mixtures thereof. They suggest that N-methylpyrrolidinone (1-methyl-2-pyrrolidinone) is “typically preferred, either alone or in combination with another aprotic, dipolar solvent.” They note that in addition to the aprotic dipolar solvent (or mixture of aprotic dipolar solvents), the solvent system may additionally comprise other solvents such as acetone, ether, hydrocarbon, toluene, benzene, and halobenzene. The reaction is said to be able to be carried out over a wide range of temperatures and pressures They suggest methyl bromide as a useful alkylating agent not requiring a pressure vessel. They further suggest that such the reactions may be carried out at a temperature somewhere in the range of room temperature (about 25° C.) to about 90° C., typically about 55° C. to about 85° C.

Of the solvents set forth in Cantrell, it has been found by the present inventors that dimethyl formamide (DMF) is particular useful in alkylation when an alkyl iodide or bromide is employed under nitrogen in the reaction scheme. Reactions are seen to be effectuated as in Cantrell from about room temperature to 90° C., without the long reaction times of weeks reported by some investigators. DMF, as opposed to the N-methylpyrrolidinone preferred by Cantrell, was found to decrease reaction times.

The present inventors have also found that addition of O-alkyl groups to the C-7 of a N-quaternary-oxymorphone compound can difficult due to elimination of the added group in the purification of crude material. The elimination may to reformation to the starting material. They have found that by reducing the 6-keto group with a reducing agent, such as sodium borohydride, elimination is significantly reduced.

Lastly, the present inventors have discovered that the R and S, axial and equatorial, stereoisomers of N-3,4-epoxy-morphinanium compounds can be easily and efficiently separated using reverse phase C-18 (length of the hydrophobic alkyl chain on the stationary phase silica) end-capped silica chromatography columns, such as a RediSep® C-18 reversed phase column. Such columns may be used with automated flash chromatography instrumentation to allow for separation of the stereoisomers—such as CombiFlash® automated flash.

### DETAILED DESCRIPTION OF THE INVENTION

In embodiments of the present invention, there is disclosed an improved method for alkylating tertiary oxymorphone compounds to their quaternary counterparts, said method comprising: dissolving the oxymorphone analog and an alkyl halide in dipolar aprotic solvent, in particular, dimethyl formamide; stirring the reaction mixture for about 2 to about 120 hours at a temperature between about 25° C. to about 90° C.; extracting the stirred reaction mixture with a non-polar solvent, such as chloroform and dichloromethane, to obtain product.

In a further embodiment of the invention there is disclosed a method for resolving R, S, axial, equatorial N-stereoisomers of oxymorphone and 3,4-epoxy-morphinanium analogs in general. Such method comprises: (a) obtaining a first composition containing a mixture of axial and equatorial N-stereoisomers of the 3,4-epoxy-morphinanium analog of interest; (b) purifying the mixture by chromatography, recrystallization, or a combination thereof to obtain a substantially pure (70% or more, more preferably 80% or more, more preferably 90% or more, yet more preferably 95% or more, and yet even more preferably 99% or more) of a diastereomeric mixture; (c) loading a diastereomeric mixture containing each of an axial or an equatorial stereoisomers onto a HPLC column and applying as a standard of at least one of the axial or equatorial stereoisomer to allow for determination of relative retention time of each stereoisomer to the other; (d) collecting the fraction determined to be the stereoisomer of interest. In a particularly useful embodiment, the HPLC system utilized is a C-18 reversed phase end-capped silica system. A useful column is the RediSep C-18 reversed phase column. Another column which has been found advantageous for the separation of the stereoisomers of such compounds is the Phenomonex Synergi Hydro-RP column (C18, 5μ, 150×4.6 mm). Conditions which may be associated with such a column are set forth below in Example 1.

### Example 1

### Exemplary HPLC Conditions for Separating N-stereoisomers of 3,4-epoxy-morphinanium Analogs

HPLC Conditions:

Hewlett Packard 1100 series:
Column: Phenomonex Synergi Hydro-RP column (C18, 5μ, 150×4.6 mm)
Flow rate: 1.0 mL/min. Column temperature: 40° C.
Detector: diode array detector monitoring @ 220 and 210 nm.
Elution: isocratic. 60% water, 30% buffer (700 ml of water, 300 mL methanol, 3 mL triethylamine and sufficient phosphoric acid to give a pH of 3.4.), 10% methanol.

Alternate HPLC Conditions:
Column: Phenomonex Synergi Hydro-RP column (C18, 5μ, 150×4.6 mm)
Flow rate: 1.5 mL/min.
Column temperature: 50° C.
Detector: diode array detector monitoring @ 220 and 280 nm.
Elution: gradient.

| Time (min.) | Methanol | Water | Mix^{a} | Curve |
|---|---|---|---|---|
| 0 | 0% | 90% | 10% | initial |
| 45 | 30% | 60% | 10% | linear |
| 45.1 | 0% | 90% | 10% | linear |
| 50 | 0% | 90% | 10% | hold |

^{a}(49.5% water, 49.5% methanol, 1% trifluoroacetic acid)

An exemplary reaction scheme using the alkylation process and separation process described above are shown in Example 2.

### Example 2

### Preparation and Isolation of (S)-17-(3,3-Dimethylallyl)-4,5α-epoxy-3,14-dihydroxy-17-methyl-6-oxomorphinanium bromide

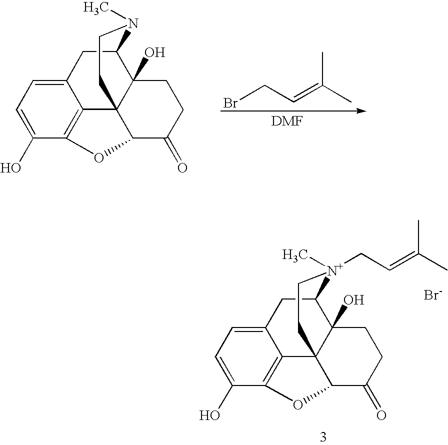

Synthetic Procedure.

Oxymorphone (200 mg, 0.66 mmol) and 3,3 dimethylallyl bromide (0.1 mL, 0.73 mmol) were dissolved in 1 mL of dimethylformamide. The reaction was stirred overnight at room temperature. The reaction was charged with additional 3,3-dimethyl allylbromide (130 mg, 0.73 mmol) and finely powdered sodium bicarbonate (18 mg, 0.21 mmol). The reaction was continued for another 24 hrs. HPLC analysis showed 74% product, 18% oxymorphone, and 8% unknown impurity. The reaction was stripped and triturated with ether. The residue was loaded onto a reverse phase chromatography column (Biotage 40 M C18) and eluted with 2 1 of a linear gradient of 0.1% trifluoroacetic acid solutions of 95:5 to 70:30 water:methanol. The product containing fractions were combined and stripped to give 100 mg of product. The residue was dissolved in water and 1 mL of a 10% solution of sodium iodide was added.

Isolation of S-Stereoisomer from R-Stereoisomer.

The aqueous phase was extracted repeatedly with 20% isopropanol in chloroform until the HPLC analysis of the aqueous phase showed less than 2% product. The combined organic phases were filtered through 1 PS paper and the solvent removed in vacuo to give 100 mg of product as a yellow solid. HPLC analysis showed the product to be 90.7% pure. The residue was then purified by column chromatography (Biotage 12M silica gel column) fluting with 760 ml of a linear gradient of 0-20% methanol in methylene chloride. The product containing fractions were combined and stripped to give 26.2 mg of product (10% yield). HPLC analysis showed the purity, to be >98%.

¹H NMR (300 MHz, CD₃OD) δ 6.75 (s, 2H), 5.66 (br t, J=6.0, 1H), 5.16 (dd, J=12.9, 6, 1H), 4.52 (dd, J=9.6, 12.9, 1H), 4.01 (d, J=4.8, 1H), 3.6-3.4 (m, 2H), 3.16-2.94 (m, 4H), 3.1 (s, 3H), 2.25 (dt, J=15, 3, 1H), 2.15-2.08 (m, 1H), 1.97 (s, 3H), 1.91 (s, 3H), 1.91-1.76 (m, 3H). MS [M⁺]: 371.2. HPLC purity: 98.3% (UV detection at 280 nm).

Detection can be carried out conveniently by ultraviolet (UV) wavelength @230 nm. Quantitation Limit is the lowest amount of an stereoisomer that can be consistently measured and reported, regardless of variations in laboratories, analysts, instruments or reagent lots. Detection Limit is the lowest amount of the stereoisomer in a sample which can be detected but not necessarily quantitated as an exact value. HPLC may be used to determine the relative amount of each stereoisomer to the other and the intermediates of the synthesis thereof by determining the area under the respective in the chromatogram produced.

In one embodiment, the chromatography is conducted using two solvents, solvent A and solvent B. Solvent A, for example, may be an aqueous solvent and solvent B may be a methanolic solvent. Further both may contain trifluoroacetic acid (TFA). In one embodiment, A is 0.1% aqueous TFA and B is 0.1% methanolic TFA. In certain embodiments the column comprises a bonded, end-capped silica. In particularly useful embodiments, the pore size of the column gel is 5 microns.

It has been found by the present inventors that the addition of an O-alkyl group at R₈ can lead to significantly different pharmacological properties

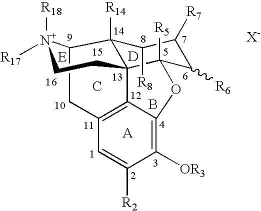

It has been found, however, that purification of such a compound is particularly difficult when the compound is an oxymorphone (i.e., R₃ is H and R₆═O). Elimination of the alkyl group appears during the purification process, causing the compound to reform to the original starting material. It has been found that reduction of the 6-keto group with a reducing agent such as sodium borohydride made the elimination much less likely. By using this approach one can gain product of sufficient purity and quantity. An example of such technique is set forth in Example 3 below.

### Example 3

### Synthesis and Isolation of (R)-17-Cyclopropylmethyl-4,5α-epoxy-3,14-dihydroxy-17-methyl-6β-hydroxy-8-propoxy-morphinanium trifluoroacetate

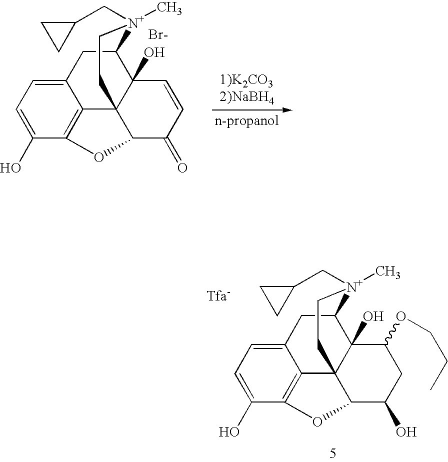

Synthetic Procedure.

A mixture of delta 7-methylnaltrexone bromide (120 mg, 0.4 mmol) and powdered potassium carbonate (1 mg, 0.07 mmol) in n-propanol was heated on a steam bath and then allowed to cool to room temperature overnight. HPLC analysis showed 13% of 8-propoxy-N-methyl naltrexone intermediate. DBU (1,8-diazabicyclo[5.4.0]undec-7-ene) 50 mg) was added and the reaction stirred and additional 4 hrs HPLC analysis showed 12% product. Additional potassium carbonate (100 mg, 0.72 mmol) was added an the reaction continued overnight at room temperature. HPLC analysis showed that the amount of intermediate had reduced to 9%. The reaction was charged with sodium borohydride (4 mg, 0.1 mmol) and stirred at room temperature overnight. In the morning another portion of sodium borohydride (4 mg, 0.1 mmol) was added and reaction was warmed in hot tap water and stirred overnight again.

Isolation of R-Stereoisomer.

The solvent was removed in vacuo and the residue dissolved in 5 ml of 0.1% trifluoroacetic acid in 95:5 water:methanol and loaded onto a reversed phase C18 column (Biotage, 40 M) eluted with a linear gradient of 95:5 to 35:65 water:methanol with 0.1% trifluoroacetic acid. The product containing fractions were combined and the solvent was removed in vacuo to give 21.4 mg of product (15% yield, 96% purity by HPLC, 90:6 ratio of isomers 6β:6α).

¹H NMR (300 MHz, CD₃OD) δ 6.77 (s, 2H), 4.86 (s, 1H), 4.42 (d, 1H), 4.04 (br d, 1H), 3.9 (dd, 1H), 3.7 (s, 3H), 3.6-3.2 (m, 4H), 3.2-2.7 (m, 5H), 2.1-1.5 (m, 6H), 1.25 (m, 1H), 0.95 (t, J=7.3, 3H), 0.85 (m, 1H), 0.65 (m, 1H), 0.48 (m, 1H). MS [M⁺]: 417.2. HPLC purity: 95.2% (UV detection at 280 nm).

### STATEMENT REGARDING EMBODIMENTS

While the invention has been described with respect to embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

1. A method for N-alkylating tertiary oxymorphone compounds with an alkyl halide said method comprising:
(a) dissolving the oxymorphone compound and an alkyl halide in dipolar aprotic solvent;
(b) stirring the reaction mixture for about 2 to about 120 hours at a temperature between about 25° C. to about 90° C.;
(c) extracting the stirred reaction mixture with a non-polar solvent to obtain product.

2. The method of claim 1 wherein the dipolar aprotic solvent is dimethyl formamide.

3. The method of claim 1 wherein the non-polar solvent is at least one of the group consisting of: chloroform and dichloromethane.

4. A method for isolating N-stereoisomers of interest from a diastereomeric mixture of a 3,4-epoxy-morphinanium, said method comprising:
(a) purifying the diastereomeric mixture by at least one of: chromatography, and recrystallization to obtain a diastereomeric mixture of at least about 90%;
(b) loading the purified diastereomeric mixture onto an eluting HPLC column;
(c) applying as a standard at least one of the N-stereoisomers of the diastereomeric mixture to said HPLC column;
(d) determining the relative retention time of each N-stereoisomer based on the retention time of the standard N-stereoisomer;
(e) collecting the fraction eluting from said HPLC column determined to be the stereoisomer of interest.

5. The method of claim 4 wherein the N-stereoisomer of interest is an R-stereoisomer.

6. The method of claim 4 wherein the N-stereoisomer of interest is an S-stereoisomer.

7. The method of claim 4 wherein the HPLC column is a C-18 reversed phase end-capped silica system.

8. A method for isolating a C-8 O-alkylated oxymorphone analog comprising:
(a) reacting the C-8 O-alkylated-oxymorphone analog with a reducing agent in a concentration sufficient to reduce the 6-keto group;
(b) applying the reduced C-8 O-alkylated-oxymorphone analog to a reverse phase HPLC column.

9. The method of claim 8 wherein the reverse phase HPLC column is an end-capped silica column.

10. The method of claim 9 wherein the end-capped silica column is C-18.
